(19) United States
(12) Patent Application Publication
(10) Pub. No.: US 2003/0100475 A1
(43) Pub. Date: May 29, 2003

(54) Predicting taxonomic classification of drug targets

(76) Inventors: Charles Pidgeon, West Lafayette, IN (US); Jeffrey A. Ruell, Malden, MA (US)

Correspondence Address: BARNES & THORNBURG, 11 SOUTH MERIDIAN, INDIANAPOLIS, IN, 46204

(21) Appl. No.: 10/117,300

(22) Filed: Apr. 5, 2002

Related U.S. Application Data
(60) Provisional application No. 60/281,749, filed on Apr. 5, 2001.

(51) Int. Cl. A61K 31/00; G06F 19/00; G01N 33/48; G01N 33/50; G01N 31/00

(52) U.S. Cl. 514/1; 702/19; 702/22

(57) ABSTRACT

Methods are provided for identifying the taxonomical classification and probable biological function of the endogenous target macromolecule of a test drug substance by comparing the membrane surface affinities of the test drug substance with compounds having a known classification.

### CROSS REFERENCE TO RELATED APPLICATION

This application claims priority under 35 U.S.C. §119(e) to U.S. Provisional Application No. 60/281,749, filed Apr. 5, 2001 expressly incorporated by reference herein.

### FIELD OF THE INVENTION

The present invention relates generally to methods for predicting the biochemical target of a compound. More particularly, the present invention relates to methods for predicting the biochemical target of a compound by measuring the membrane binding properties of the compound.

### BACKGROUND OF THE INVENTION

There continues to be a significant development effort directed to the development of research tools and/or protocols for enhancing the efficiency and efficacy of drug discovery research. The goal of such efforts has been to define efficient methodologies for predicting not only biological activity, but the pharmacokinetic properties of putative drug substances critical to their therapeutic efficacy as well. In the past drug leads have been generated by comparing their structural data, biological and physical properties with those of known compounds having recognized biological activity in vivo. There has been developed a significant body of literature directed to the development of drug discovery protocols designed no only to predict in vitro biological activity, but to predict pharmacokinetic properties based on comparison of physical, chemical and biological descriptors and the use of pattern recognition analysis of such descriptors.

With the advent of combinatorial chemistry and other techniques to generate a range of putative drug compounds, there continues to be a need for the development of high throughput methods and systems for predicting the biological targets for such molecules as well as for predicting pharmacokinetic properties and providing guidance for drug design to improve pharmacokinetic properties. Such methods would also be advantageous for screening the large libraries of compounds that most pharmaceutical companies have amassed over the years.

### SUMMARY OF THE INVENTION

The present invention is based at least in part on the discovery that known drug substances, necessarily exhibiting pharmacokinetic properties for therapeutic efficacy, exhibit affinities for various membrane mimetic surfaces at levels within a fairly well defined range. Furthermore, compound structures, the taxonomic classification of the endogenous target molecule, and the art-recognized individual pharmacokinetic properties are all closely correlated with a drug's relative affinities for two or more membrane-like or membrane mimetic surfaces.

The present invention provides methods for predicting the taxonomic classification of putative or test drug substances. Taxonomic classification of a drug substance is the class of endogenous macromolecular targets that the drug substance is most likely to interact with. Examples of taxonomic classification are, but not limited to, G-protein linked receptors, ligand gated ion channel receptors, intracellular receptors, ion channels, enzyme inhibitors, ion transport inhibitors, neurotransmitter transport inhibitors, and molecules that bind to DNA. In one aspect of the present invention, the methods for predicting taxonomic classification comprise the steps of determining the membrane affinities of a group of known drug substances (referred to herein as the “control compounds”) for at least two different membrane surfaces wherein the taxonomic classification of the control compounds are known, producing a calibration curve from the control compounds' affinities for the membrane surfaces, determining the affinity at least one test drug substance for the membrane surfaces wherein the taxonomic classification is unknown, and finally, determining the taxonomic classification of the test drug substances by comparing the membrane affinities of the compound to the calibration curve. It will be appreciated that chemical substances having a known taxonomic classification, although not known as drug substances, can also be used in the methods of the present invention.

In another aspect of the invention, any membrane system can be used to measure membrane affinities of the control compounds as long as the membrane affinity measurement correlates to the taxonomic classification of the control compounds. Non-limiting examples of membrane systems for measuring affinity are Langmuir-Blodgett films, liposomes, micelles, and membrane mimetic surfaces. In a preferred aspect of the invention, at least one of the membrane surfaces is neutral and at least one of the surfaces is negatively charged. More preferably, the membrane surfaces are immobilized artificial membranes (IAM). Immobilized membranes allow for rapid and facile determination of the membrane surface affinities of a drug substance.

In another aspect of the present invention, the methods for predicting taxonomic classification comprise the steps of determining the membrane affinities of a group of control compounds for at least two different membrane surfaces wherein the taxonomic classification of the control compounds are known, producing a calibration curve from the control compound affinities for the membrane surfaces, determining the affinity of at least one test drug substance for the membrane surfaces wherein the taxonomic classification is unknown, finding the control compounds with the closest structural similarity to the test drug substance by comparing the molecular descriptors of the test drug substance to those of the control compounds, and finally, determining the taxonomic classification of the test drug substances by comparing the membrane affinities of the test drug substance to the calibration curve and the taxonomic classifications of the control compounds with the closest similarity to the test drug substance based on the molecular descriptors. Non-limiting examples of molecular descriptors are number of donor atoms for H-bonds, number of acceptor atoms for H-bonds, the sum of the atomic polarizability, hydrophilicity, hydrophobicity, number of rings, number of aromatic/saturated rings, the position of the rings in relation to one another, the presence of halogenated group on aromatic rings, presence of an amino group, position of amino groups in the molecule and mean atomic van der Waals volume. The use of the molecular descriptors with membrane surface affinities results in improved prediction of taxonomic classification as compared to the use of membrane affinities alone.

Additional objects, advantages, and features of the present invention will become apparent from the following description, taken in conjunction with the accompanying drawings and appended claims.

### BRIEF DESCRIPTION OF THE DRAWINGS

The various advantages of the present invention will become apparent to one skilled in the art by reading the following specification and by referencing the following drawings in which:

FIG. 1 is a graph presenting membrane affinity values forming at least a portion of a data set for use in drug discovery protocols in accordance with this invention. Each point on the graph represents the membrane affinity values for a drug compound member of the data set used in accordance with this invention and represents the relative binding of said compound to a membrane mimetic surface comprising phosphatidyl ethanolamine (X-axis) and another membrane mimetic surface comprising phosphatidyl serine (Y-axis);

FIG. 2 is similar to FIG. 1 except that only drug substances exhibiting enzyme inhibition activity are represented;

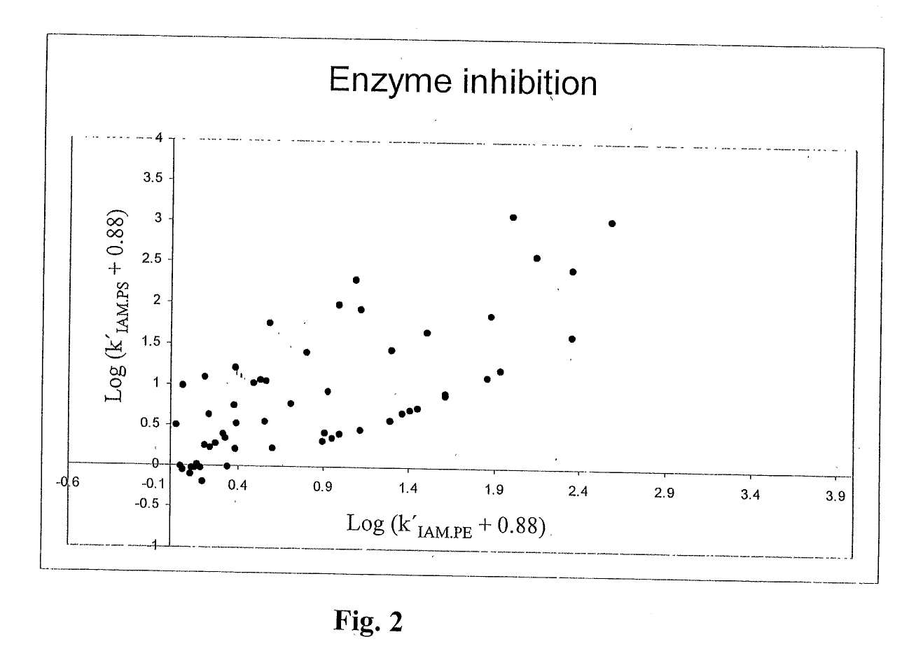

FIG. 3 is similar to FIG. 1 except that only drug substances exhibiting activity through G-protein linked receptors are represented;

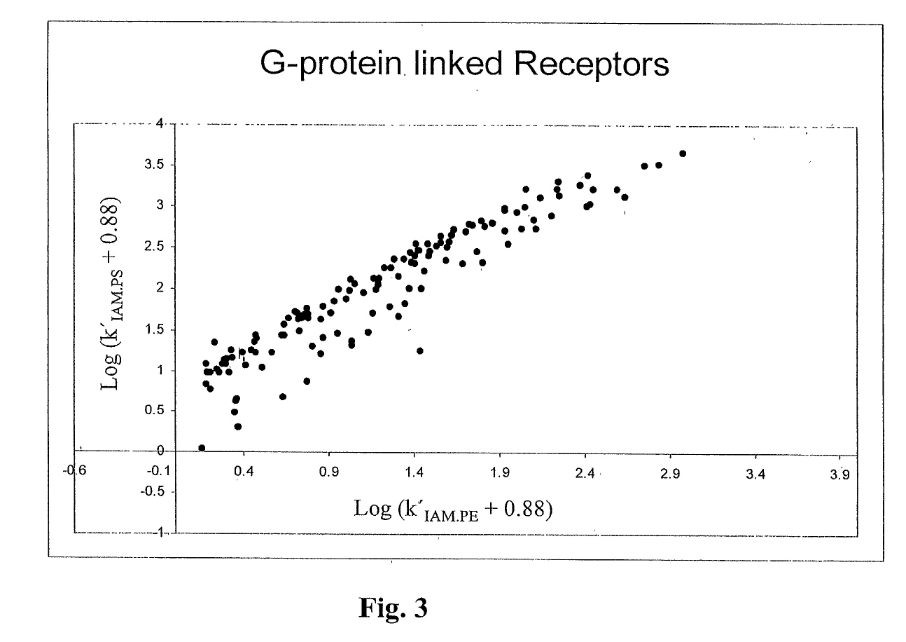

FIG. 4 is similar to FIG. 1 except that only drug substances interacting with intracellular receptors are represented;

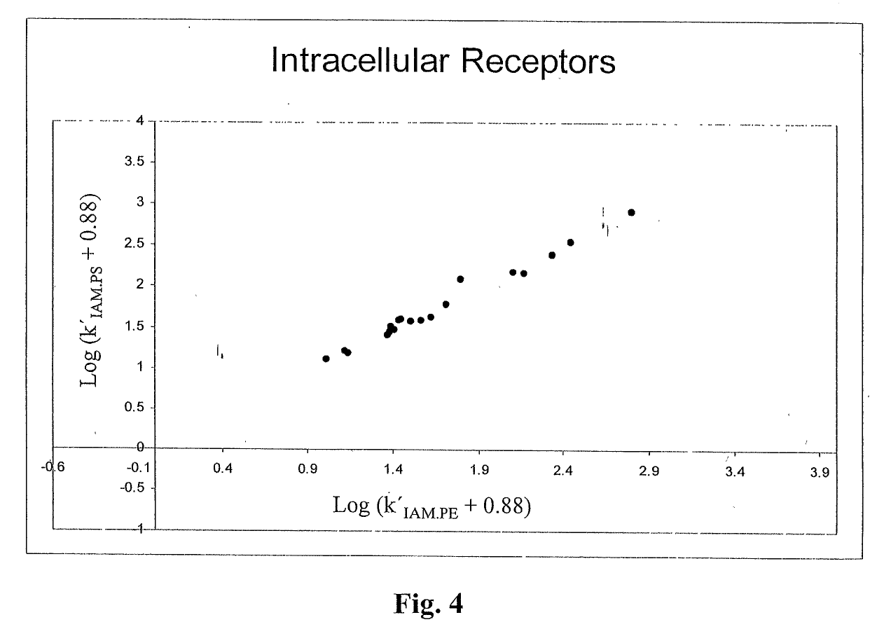

FIG. 5 is similar to FIG. 1 except that only drug substances interacting with ion channels are represented;

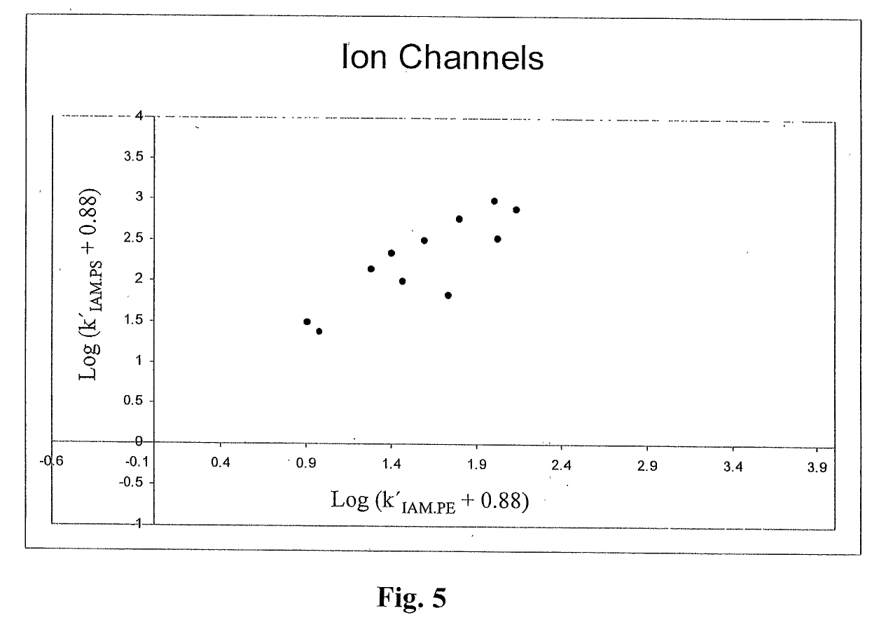

FIG. 6 is similar to FIG. 1 except that only drug substances exhibiting ion transporter inhibition activity are represented;

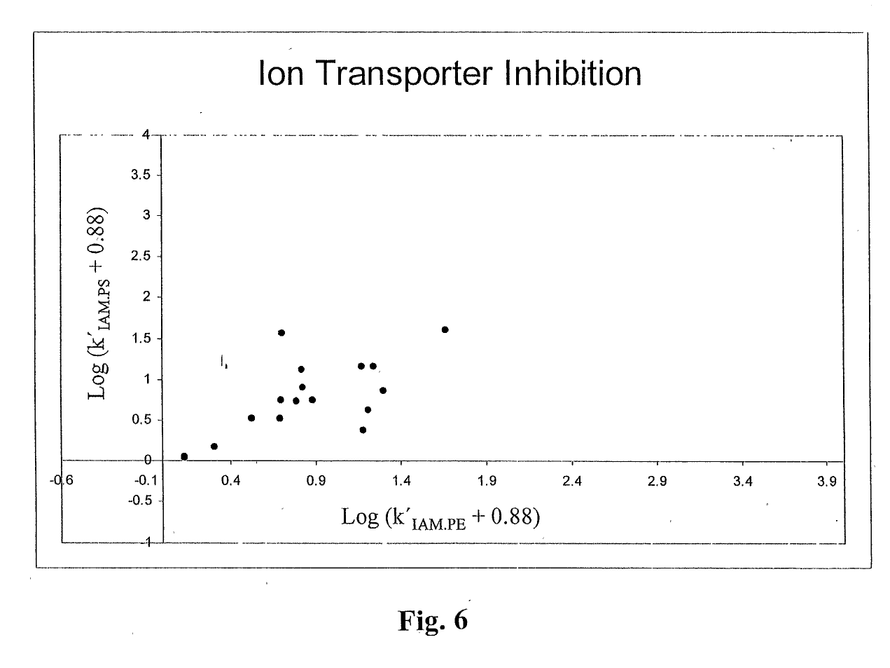

FIG. 7 is similar to FIG. 1 except that only drug substances interacting with ligand-gated ion channels are represented;

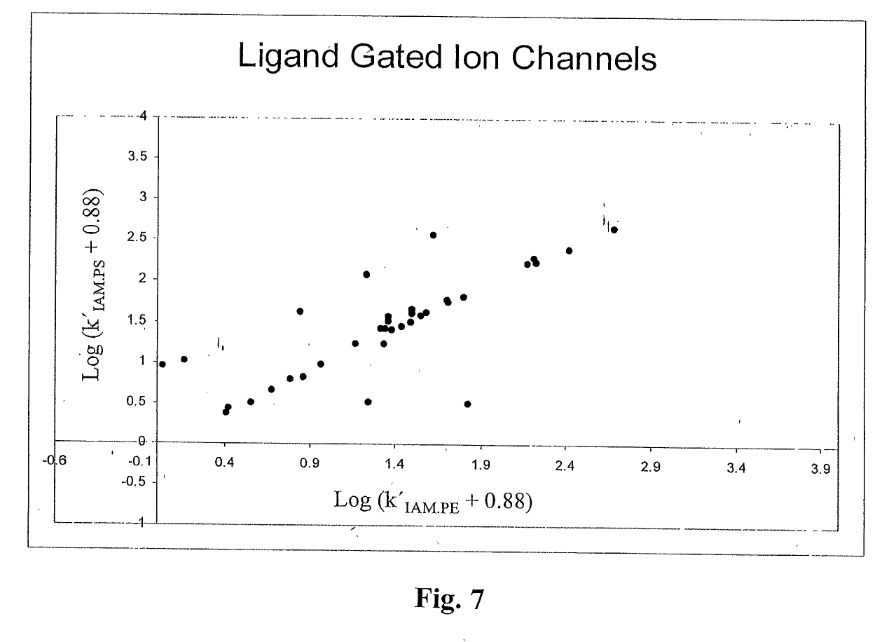

FIG. 8 is similar to FIG. 1 except that only compounds exhibiting neurotransmittor re-uptake inhibition activity are represented;

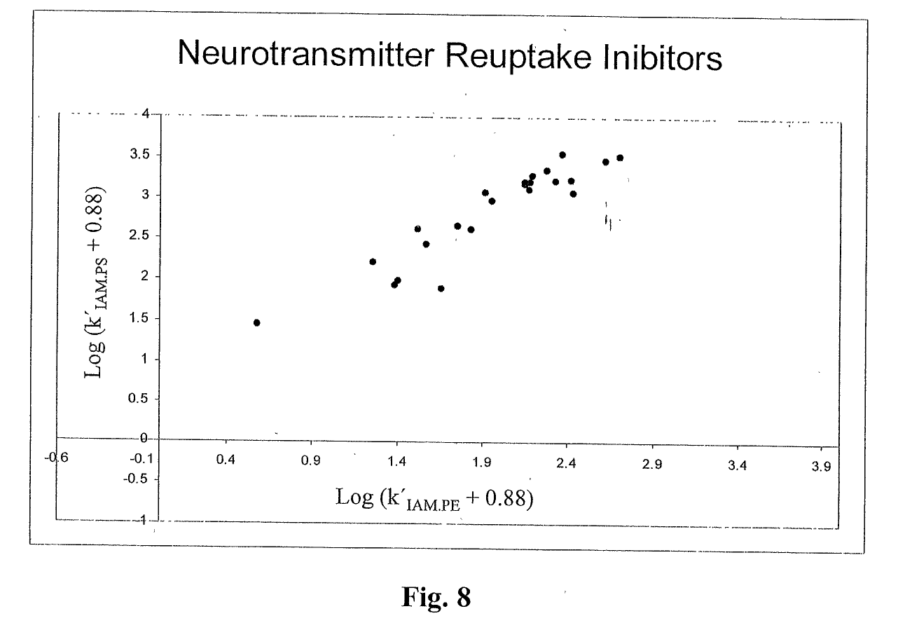

FIG. 9 is similar to FIG. 1 except that only compounds exhibiting interaction with nucleic acids are represented; and

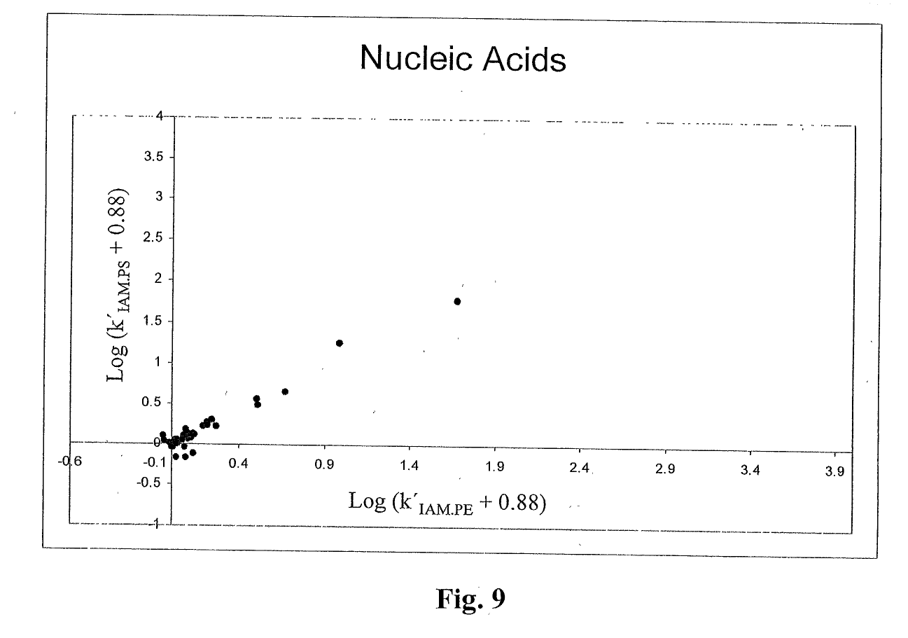

FIG. 10 is a graph presenting a 2-dimensional PCA plot of approximately 400 commercial drug substances using four membrane binding constants.

### DETAILED DESCRIPTION OF THE INVENTION

The present invention is based at least in part on the discovery that known drug substances, necessarily exhibiting pharmacokinetic properties for therapeutic efficacy, exhibit affinities for various membrane mimetic surfaces at levels within a fairly well defined range. Furthermore, compound structures, the taxonomic classification of the endogenous target molecule, and the art-recognized individual pharmacokinetic properties are all closely correlated with a drug's relative affinities for two or more membrane-like or membrane mimetic surfaces.

The present invention provides methods for predicting the taxonomic classification of putative or test drug substances. Taxonomic classification of a drug substance is the class of endogenous macromolecular targets that the drug substance is most likely to interact with. Examples of taxonomic classification are, but not limited to, G-protein linked receptors, ligand gated ion channel receptors, intracellular receptors, ion channels, enzyme inhibitors, ion transport inhibitors, neurotransmitter transport inhibitors, and molecules that bind to DNA. In one aspect of the present invention, the methods for predicting taxonomic classification comprise the steps of determining the membrane affinities of a group of known drug substances (referred to herein as the “control compounds”) for at least two different membrane surfaces wherein the taxonomic classification of the control compounds are known, producing a calibration curve from the control compounds' affinities for the membrane surfaces, determining the affinity at least one test drug substance for the membrane surfaces wherein the taxonomic classification is unknown, and finally, determining the taxonomic classification of the test drug substances by comparing the membrane affinities of the compound to the calibration curve. It will be appreciated that chemical substances having a known taxonomic classification, although not known as drug substances, can also be used in the methods of the present invention.

In another aspect of the invention, any membrane system can be used to measure membrane affinities of the control compounds as long as the membrane affinity measurement correlates to the taxonomic classification of the control compounds. Non-limiting examples of membrane systems for measuring affinity are Langmuir-Blodgett films, liposomes, micelles, and membrane mimetic surfaces. In a preferred aspect of the invention, at least one of the membrane surfaces is substantially neutral and at least one of the surfaces is negatively charged. More preferably the substantially neutral membrane is based on phosphatidylethanolamine (PE), phosphatidylcholine (PC) or sphingomylin (SM) and the negatively charged membrane on phosphatidylserine. More preferably, the membrane surfaces are immobilized artificial membranes. Immobilized membranes allow for rapid and facile determination of the membrane surface affinities of a drug substance. More preferably the immobilized membranes are ^{ester}IAM.PE^{C10/C3}, ^{ester}IAM.PS^{C10/C3}, ^{ester}IAM.PC^{C10/C3}, or IAM.SM^{C10/C3} columns.

In another aspect of the present invention, the methods for predicting taxonomic classification comprise the steps of determining the membrane affinities of a group of control compounds for at least two different membrane surfaces wherein the taxonomic classification of the control compounds are known, producing a calibration curve from the control compound affinities for the membrane surfaces, determining the affinity of at least one test drug substance for the membrane surfaces wherein the taxonomic classification is unknown, finding the control compounds with the closest structural similarity to the test drug substance by comparing the molecular descriptors of the test drug substance to those of the control compounds, and finally, determining the taxonomic classification of the test drug substances by comparing the membrane affinities of the test drug substance to the calibration curve and the taxonomic classifications of the control compounds with the closest similarity to the test drug substance based on the molecular descriptors. Non-limiting examples of molecular descriptors are number of donor atoms for H-bonds, number of acceptor atoms for H-bonds, the sum of the atomic polarizability, hydrophilicity, hydrophobicity, number of rings, number of aromatic/saturated rings, the position of the rings in relation to one another, the presence of halogenated group on aromatic rings, presence of an amino group, position of amino groups in the molecule and mean atomic van der Waals volume. The use of the molecular descriptors with membrane surface affinities results in improved prediction of taxonomic classification as compared to the use of membrane affinities alone.

A significant aspect of the present invention is that knowledge of the structure of a compound is not necessary. Thus, it is possible to screen complex mixtures of unknown compounds (e.g. natural product extracts) and predict the taxonomic classification of the test compounds and therefore allow the skilled artisan to predict the therapeutic potential of the test compounds. If nascent therapeutic targets were used to obtain similar information, the therapeutic targets would need to be isolated, or acquired, reconstituted and radioactivity or some other detection method used for the analysis.

It will also be appreciated that there is no restriction on the number of membrane surfaces that can be used and subsequently, if immobilized membranes are being used, no restriction on the number of chromatography surfaces that can be used. Chromatographic retention times depend on solute structure, the chromatographic surface, and the mobile phase. The mobile phase and chromatography column sizes are constant during the data collection to assure that the interaction of the compounds with the immobilized membranes is the only variable. Elution times can be decreased by increasing the flow rate, thus keeping the elution volume (a measure of affinity) the same. The difference between the columns should only be the membrane surface ligand that is attached to the solid support. Volume, the bonded phase thickness of the solid surface, the volume of the immobilized ligand and the hydrophobic environment, by way of non-limiting examples, should be approximately the same in all columns.

The present invention makes use of the discovery of a significant and surprising level of correlation between empirically determined values relating to the level of affinity for two or more membrane or membrane mimetic surfaces, with both the chemical structure and the taxonomic classification of the compound exhibiting such relative membrane affinities. Thus important to implementation of the present invention is the preparation and utilization of a data set including structural and empirical information for known drug substances (and, with advantage, for other compounds as well) wherein the empirical data includes values relating to the affinity of the respective compound represented in the data set for two or more surfaces, typically membrane mimetic surfaces and a value relating to the nature (i.e., the taxonomic classification) of the endogenous macromolecular target, if known, of the drug substances represented in the data set. In one embodiment the data set is stored electronically in computer accessible form/format as an array of values associated with each compound member of the data set. The chemical structural data for the respective compounds can be stored in either a two-dimensional or three-dimensional format accessible and searchable using commercially available search software capable of identifying those chemical structures in the data set exhibiting some predefined degree of structural/substructural similarity with a test compound.

The numeric values characteristic of membrane affinity for use in forming the data set can be determined by any of a wide variety of art-accepted techniques. In one embodiment of this invention the numeric values characteristic of membrane affinity are determined chromatographically using an aqueous mobile phase and a stationary phase comprising a membrane mimetic surface, for example, in a high performance liquid chromatographic system such as that described in U.S. Pat. No. 4,931,498, expressly incorporated herein by reference. The term membrane or “membrane mimetic surface” as used in describing and defining the present invention, refers to any surface bearing amphiphilic molecules (i.e., those having both lipophilic and hydrophilic portions capable of exhibiting some selective affinity or otherwise interacting with a solute), for example, a test or control compound in a fluid phase in contact with the surface. The term is intended to encompass abroad scope of commercially available stationary phases detailed for use in chromatographic applications. Preferred membrane mimetic surfaces are those described in the above-incorporated U.S. Pat. No. 4,931,948.

Thus, in its broadest scope, the present invention is directed to use of a data set comprising chemical structural and empirical data for known drug substances, i.e., compounds proven safe and effective for therapeutic use. Alternatively, the data set may comprise any chemical molecule that is known to interact with a biological target and thus has a taxonomic classification. The empirical information for the drug compound members in the data set typically includes a value characteristic of the relative affinity exhibited by the compound for at least two unique membrane or membrane mimetic surfaces, preferably at least one of which is a substantially neutral surface and the other of which is a negatively charged surface (under the conditions of measured membrane affinity). The data array for the drug compound members of the data set can, and preferably does, include values indicative of pharmacokinetic and/or pharmacodynamic properties of at least a portion of the drug members of the data set. In one embodiment the data set includes as well as value relating to the nature, more particularly the taxonomic classification, of the endogenous molecule known to be the target of the respective data set drug substance member.

The data set is used, typically stored electronically in a computer readable format and used in systems and methods for drug discovery research. If the structure of the test compound is known, the chemical structures in the data set can be searched using any one of several commercially available structure similarity search programs to identify the chemical structures of compounds in the data set exhibiting some predetermined degree of similarity with the test compound. Alternatively, if the structure of the test compound is not known, its relative affinities to at least two of the membrane mimetic surfaces, represented by the numeric values in the data set, can be measured empirically, for example, by chromatographic analysis, and the empirically determined membrane affinity values for the test compound or drug substance can be used as the basis of a search of the data set for data set drug compound members exhibiting similar membrane binding characteristics. Preferably both the membrane affinity and structural similarity variables are used together to predict the taxonomic classification of the unknown compound.

In another embodiment the present invention also provides a system for using the data set and data set correlations to predict taxonomy of potential endogenous target molecules. The system typically includes a data storage device having the data set in computer readable format, a data entry device for entering the chemical structure or the membrane affinity values for the test compound, and a programmable microprocessor communicating with the data entry device and the data storage device and programmed to search the data set to identify drug compound data set members most structurally similar to, or membrane binding characteristics most similar to, that/those of the test compound. Typically the system includes as well an output device in communication with the microprocessor for reporting the results of the data set search.

The present invention provides a powerful tool for drug discovery research. It enables the prediction of the nature of the endogenous target molecules with which a putative test drug compound is most likely to interact. The predictions can be based on the chemical structure of the compound, if known, or on membrane affinity values determined empirically for the test compound. The data set used in carrying out this invention thus provides a means for correlating the chemical structures and taxonomic classification of macromolecular targets with values for relative affinities of the data set compounds for two or more membrane surfaces. Preferably the data set includes values relating to the affinity of the respective data set members for at least one substantially neutral membrane surface and another exhibiting a negative charge at physiological pH.

The present invention provides a system for predicting taxonomy of potential endogenous target macromolecules of a test drug substance of known chemical structure using a membrane affinity based correlation of the chemical structures of known drug substances, their endogenous target macromolecules, and their empirically defined chemical structure/membrane affinity relationships. The system comprises a data storage device having in computer readable format a data set comprising chemical structures of a multiplicity of control compounds comprising drug substances, and for each compound, numeric values relating to the affinity of said compounds to at least a negatively charged membrane surfaces and a neutral membrane surface, and a value relating to the identity or function of the known endogenous macromolecular target, if any, of said compound. It also includes a data entry device for entering the chemical structure or membrane binding data of the test drug substance in computer readable format and a programmable microprocessor in communication with said data entry device and said data storage device, said microprocessor programmed to compare the chemical structure of the test drug substance entered into the data entry device with chemical structures in the data set to identify the chemical structures in the data set having a predetermined degree of similarity with the structure of the test drug substance. Typically the system also includes an output device in communication with the microprocessor and capable of reporting, upon user request, the chemical structures or other identification of control compounds having the predetermined degree of similarity with the chemical structure of the test drug substance, and other data stored for said identified control compound(s).

In one embodiment there is provided a method for predicting taxonomy of potential endogenous target macromolecules for a test drug substance of known chemical structure. The method comprises the steps of selecting a database including, in computer readable format, chemical structures for a multiplicity of control compounds, said control compounds comprising known drug substances each having a known endogenous target macromolecule, an for each known drug substance, a value corresponding to the taxonomy of its known endogenous target macromolecule, searching the database for compounds having a chemical structure similar to that of the test drug substance and identifying those control compounds that have a predetermined degree of similarity to the test drug substance, identifying the taxonomy of the endogenous target molecule of the control compounds having the predetermined degree of structural similarity, if any, to the test drug substance, and if no compounds are identified as having the predetermined degree of structural similarity, repeating the searching step using a lower predetermined degree of similarity until at least one compound in the database is identified, and using the taxonomy of the identified control compound(s) to predict the taxonomy of the target macromolecule of the test drug substance. Preferably the database further comprises in computer readable format, numeric values relating to the affinity of each control compound to at least a negatively charged membrane mimetic surface and/or numeric values relating to the affinity of each control compound to at least a negatively charged membrane surface and a neutral membrane surface.

The control compounds in the database can further comprise compounds not known to be drug substances but compounds for which is known and stored in the database numeric values relating to their respective relative affinities for at least a negatively charged membrane surface and a neutral membrane surface. It can occur in implementing the method that at least one of the control compounds identified to have the predetermined degree of structural similarity to the test drug substance is a compound for which the taxonomic classification is not known. In that case the method further comprising the step of identifying the control compound or compounds in the database for which the taxonomic classification is known and which has membrane binding properties most similar to the identified compound for which the taxonomic classification is not known. In that regard the method can further comprise the step of displaying an array (X_{A}, Y_{B}) for each of at least a subset of the control compounds in the database, wherein X_{A} is the numeric value relating to the affinity of the control compound for one membrane surface and Y_{B} is the numeric value relating to the affinity of the control compound for a second membrane surface, where said subset of control compounds includes the compounds identified to have the predetermined degree of structural similarity to the test drug substance.

In a related aspect of the invention a method is provided for identifying the taxonomical classification and probable biological function of the endogenous target macromolecule of a test drug substance. The method comprises the steps of identifying two or more membrane surfaces including a first membrane surface having a negatively charged surface and a second substantially neutral membrane surface, identifying a set of control compounds comprising drug substances having known endogenous macromolecular targets, defining for each control compound a numeric value related to its affinity for each membrane surface, defining for the test drug substance a numeric value related to its affinity for each membrane surface, for each taxonomical target macromolecule classification, identifying a subset of control compounds having that same or similar target macromolecules and establishing a correlation between said taxonomical target macromolecule classification and membrane affinity values for the control compounds exhibiting said values, comparing the membrane affinity related numeric values for the test drug substance with the taxonomy correlated membrane affinity values for the control compounds, and selecting the taxonomical classification(s) that best match the membrane affinity values for the test drug substance.

Similarly a method is enabled for identifying the taxonomical classification and probable biological function of the endogenous target macromolecule of a test drug substance. The method comprises the steps of identifying two or more membrane surfaces including a first membrane surface having a negatively charged surface and a second substantially neutral membrane surface, identifying a set of control compounds comprising drug substances having known endogenous macromolecular targets, defining for each control compound a numeric value related to its affinity for each membrane mimetic surface, defining for the test drug substance a numeric value related to its affinity for each membrane surface, comparing the membrane affinity related values for the test drug substance with those for the control compounds and identifying those control compounds having membrane affinity related values similar to those of the test compound, and identifying the taxonomical target macromolecule for those control compounds having membrane affinity values similar to those of the test drug substance.

The foregoing and other aspects of the invention may be better understood in connection with the following examples, which are presented for purposes of illustration and not by way of limitation.

### EXAMPLE 1

### Generation of a Calibration Curve Based on MAFs

The k′ values described in the example characterize membrane binding constants of drug substances measured on immobilized artificial membrane (IAM) chromatography surfaces. In the present invention, k′ values are used interchangeably with membrane binding constant. Membrane binding constants are only one group of parameters that characterize membrane-solute interactions. Other parameters can also be used, such as, but not limited to, interfacial pKa, membrane enthalpy, and the on-off kinetics of solutes from membrane surfaces.

Four k′ values were experimentally obtained of approximately 400 commercial drug substances using the methods of U.S. Pat. Nos. 4,931,498 and 4,927,879, expressly incorporated by reference. See Appendix A. A few non-drug substances were also evaluated, but most compounds in the database used to enable the present invention are commercial drugs. The LAM surfaces used for obtaining the k′ values of the approximately 400 compounds include ^{ester}IAM.PE^{C10/C3}, ^{ester}IAM.PS^{C10/C3}, ^{ester}IAM.PC^{C10/C3}, and IAM.SM^{C10/C3}.

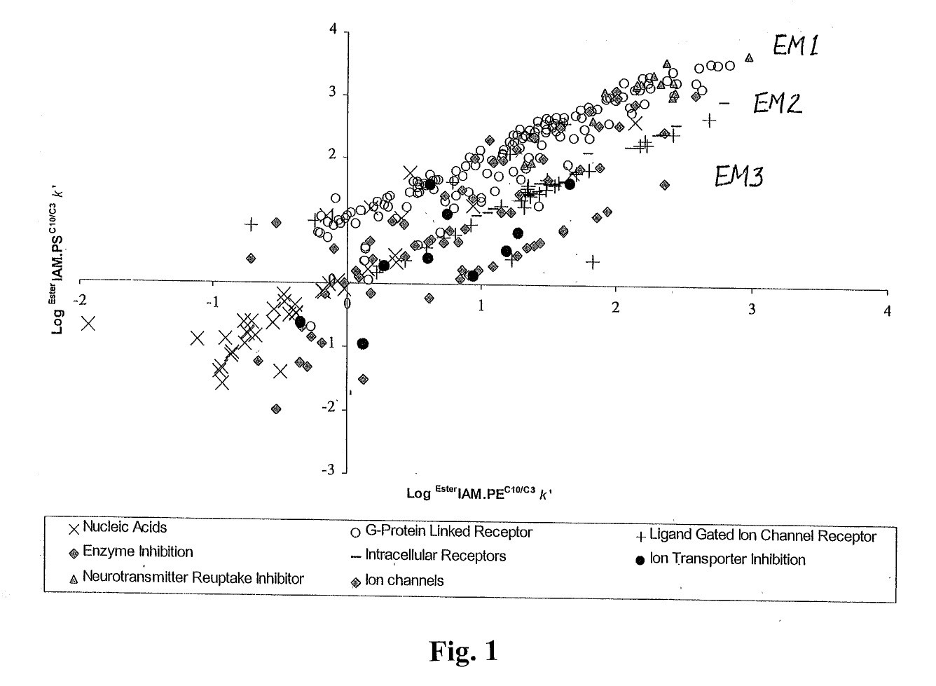

FIG. 1 is a graphical representation of the efficacy mechanisms (EM) whereby actual membrane binding data (MAFs) are plotted for two IAM surfaces and the EM regions are written on the areas where compounds would cluster for that group. Efficacy mechanisms are compound properties that are controlled by the structure of the compound and modeled by the present invention. The two IAM surfaces were ^{ester}IAM.PE^{C10/C3} and IAM.PS^{C10/C3}. FIG. 1 shows three EM regions represented by three approximately linear lines, with a few compounds residing between the EM-1 and EM-2 lines. A log₁₀ function was applied to each MAF variable because of the dynamic range of the data and especially because values were concentrated at the low end of th dynamic range. Relationships between EMs, taxonomic classification and MAF variables could be seen more clearly when the variables were a log₁₀ function.

It was also found that MAF variables from only one IAM could not discriminate taxonomic classification. However, the use of log₁₀ MAF variables from at least two IAMs were effective for distinguishing taxonomic classification. The preferred combinations were a negatively charged IAM (^{ester}IAM.PS^{C10/C3}) and a substantially neutral IAM (^{ester}IAM.PE^{C10/C3}, ^{ester}IAM.PC^{C10/C3}, or IAM.SM^{C10/C3}). The EM plot shown in FIG. 1 was obtained by plotting, on a logarithmic scale, the raw membrane binding data and consequently, the numerical values of the {x,y} coordinate system have physical significance. The physical significance is that the x coordinate provides the number of column volumes needed to elute the compound from an ^{ester}IAM.PE^{C10/C3} column, and the y coordinate provides the same information about the compound for an ^{ester}IAM.PS^{C10/C3} column. The ^{ester}IAM.PS^{C10/C3} is a negatively charged chromatographic surface whereas ^{ester}IAM.PE^{C10/C3} is approximately a neutral surface. Thus, basic compounds will tend to reside near the EM-1 line, neutral compounds near the EM-2 line and acidic compounds near the EM-3 line shown in FIG. 1.

The majority of the compounds in the EM-1 region are small molecule compounds that bind to G-coupled proteins and neurotransmitter reuptake inhibitors. In contrast, the EM-2 region tends to cluster neutral compounds which bind to ligand gated ion channels, enzymes, nucleic acid targets, and intracellular receptors. Finally, the EM-3 region tends to cluster acidic compounds which bind to ion channels. Ion transport inhibitors are scattered across all three lanes.

### EXAMPLE 2

### Results Using MAFs Alone or with Molecular Descriptors

Two public domain classification methods were used to predict the compound taxonomic classification, which is a categorical property. The classification algorithms were nearest neighbor classifiers (NNC) and kernel classifiers (KC). These algorithms can find highly nonlinear relationships between MAF values and taxonomic variables. Cross validation studies were performed using these predictive algorithms. Cross validation means that one compound is withheld from the calibration data set or curve, and the compound, with known categorical properties (i.e. taxonomic classification) is classified. This is redone for every compound in the data set. MAFs alone generated a 62% success rate in classifying all the compounds (about 320) used in the study. When molecular descriptors were used the classification rate increased to 70%. The molecular descriptors used in this study were number of donor atoms for H-bonds, number of acceptor atoms for H-bonds, sum of atomic polarizability, hydrophilic factor, number of rings, and mean atomic van der Waals volume. In comparison, random classification would be expected to correctly predict the taxonomic classification of only 10% of the compounds.

### EXAMPLE 3

### Taxonomic Classification Using Decision Trees

The data were also analyzed by applying commercial decision tree software, CART from California Statistical Software, Inc. These results also show that MAF values plus molecular descriptors gave better classification (75%) than MAF measurements alone (58%) or molecular descriptors alone (63%). All cases were used in generating rules for the learning tree. Cross validation was performed by dividing the population into roughly ten equal parts, taking nine of the ten parts in turn to generate a test tree and using the remaining one part for error estimation. In addition to classification, decision tree algorithms also provide prediction rules as branchings and probabilities. Such could be very useful when dealing with new compounds.

### EXAMPLE 4

### General Experimental Procedures

A Bruker-Esquire LC/MS system (LC HP 1100 series) interfaced with an Gilson 235P autoinjector, and equipped with an orthogonal electrospray ionization (ESI) source and an ion trap mass analyzer was used for the collection of MS data. Single injection UV data collection was carried out on a HP 1100 series HPLC. All chromatographic data was collected with 15% acetonitrile in 0.01M PBS buffer (pH 7.4) as the mobile phase. Unless otherwise indicated, the mobile phase flow was programmed to follow a stepped gradient: constant flow rate of 0.5 mL/min for the first 10 minutes of data collection; the flow rate was then stepped to 4 mL/min over 20 minutes, after which time the mobile phase flow was held at a constant 4 mL/min. All IAM columns were subjected to a performance test prior to use, to ensure high quality and reproducibility in the chromatographic data. The column void volume (V₀) was also established for each column, as part of quality control.

All samples were obtained from commercial sources. All chemicals and solvents were of analytical grade and were used without further purification.

IAM columns: Membrane Affinity Fingerprints (MAFs) were determined on the following membrane mimetic surfaces: ^{ester}IAM.PC^{C10/C3}, ^{ester}IAM.PE^{C10/C3}, ^{ester}IAM.PS^{C10/C3}, IAM.SM^{C10/C3}, which were synthesized under strict QC according to known methods (see PCT/US98/17398 published as International Publication WO 99/10522, incorporated herein by reference). The stationary phase material (5 μm particle size, 80 Å pore size) was packed into 4.6×30 mm columns by Column Engineering, Ontario, Calif. All other experimental procedures were as disclosed in U.S. Pat. Nos. 4,931,498 and 4,927,879 and PCT Application No. WO 99/10522, all expressly incorporated by reference.

Data analysis: The structure search routines were performed with CS ChemFinder 4.0, commercially available software capable of performing chemical structure/substructure similarity searches. Briefly, some of the compound information stored in the integrated database was retrieved (including, but not limited to, 2D structure, molecular weight, compound name) and stored in a file format searchable by ChemFinder. Substructure searches and/or complete structure similarity searches were conducted to identify compounds in the database that had structural similarity with the test compound, the ADME properties of which needed to be assessed. Other commercially available software for structure similarity searching can be substituted for ChemFinder for use in identifying compounds in the database having some predetermined degree of structural similarity with a test compound.

ChemFinder can perform exact structure/substructure searches and complete structure/substructure similarity searches. An exact search is based on atom connectivity comparison. The program compares the types of atoms and the order and way (bond type) in which they are connected, in the query and target molecules. If some atoms or bonds are missing, added, or different, the query structure and the target structure do not match. An exact search may be conducted with a complete compound structure or a substructure as query.

Similarity searches on the other hand rely instead on the notion of molecular descriptors. Each molecule, or portion of molecule (substructure), can be represented as a collection of molecular descriptors. ChemFinder uses a large number of descriptors. In the case of a complete structure similarity, the algorithm compares the number of descriptors the query and target molecules have in common to the number of descriptors they have in total. The ratio of these two values is called the Tanimoto coefficient, i.e., the similarity ratio. For substructure similarity searches, the concept is similar: the algorithm determines what percentage of descriptors in the query molecule are also present in the target. This value is the substructure similarity ratio.

ChemFinder structure searches were conducted in the MAF database using a compound of unknown taxonomic classification as query structure. The types of searches included exact substructure searches and structure similarity searches. For each test compound, the hits generated by the various ChemFinder structure searches were evaluated. A group of structures (typically 3-6), which showed the highest degree of similarity with the query compound, were selected from the resulting structure search hit list. The structure selection was conducted according to structural and chemical criteria. These criteria included (but are not limited to) acid/base chemistry, number of heteroatoms, polarity, number of rings, lipophilicity, topology and size of the molecules. The membrane affinities of the selected MAF database compounds were then compared to the compound of unknown taxonomic classification and a taxonomic classification assigned to the unknown.

### EXAMPLE 5

### Membrane Affinity Based Array

Previous work has established the experimental methods for obtaining membrane binding information for the present invention. See, for example, published PCT Application No. WO 99/10522 which describes the methods for obtaining k′ values, and other parameters, that are used in practice of the present invention.

The k′ values described in the examples below characterize membrane binding constants of solutes measured on immobilized artificial membrane (IAM) chromatography surfaces. For the purpose of the invention, membrane binding constant and k′ are considered synonymous. Membrane binding constants are only one group of parameters that characterize membrane-solute interactions. Other parameters include the interfacial pKa, membrane enthalpy, the on-off kinetics of solutes from membrane surfaces, etc. Thus, although k′ values are described in the examples, the invention is not limited to only parameters that characterize equilibrium binding between solutes and membranes.

Four k′ values were experimentally obtained of approximately 400 commercial drug substances listed in Appendix A. A few non drug-substances were also evaluated, but most compounds in the database used to enable the present invention are commercial drugs. The IAM surfaces used for obtaining the k′ values of the ˜400 compounds include: ^{ester}IAM.PC^{C10/C3}, ^{ester}IAM.PE^{C10/C3}, ^{ester}IAM.PS^{C10/C3}, IAM.SM^{C10/C3}.

A database containing four membrane binding parameters for ˜400 compounds is a 400×4 matrix. Because space is 3 dimensions, four parameters can not be plotted without a reduction in the number of variables. Principle component analysis (PCA) is an established method for viewing N-dimensional data when the data exceeds 3 parameters, i.e., when the number of parameters exceeds a 3 dimensional {x, y, z} coordinate system. Briefly, PCA calculates a new coordinate system that is mean centered to the data being analyzed. Most important, PCA plots of N-dimensional data show a coordinate system that maximizes the variance in the data. In other words, PCA is an established method for viewing the maximum separation of individual N-dimensional data points in 3 or less dimensions. In essence, PCA provides a new coordinate system, derived from the data itself, such that when the data is plotted in PCA coordinate space a maximum separation of the data is obtained. The dimensions (or axes) are sequentially denoted as principle component 1, principle component 2, and principle component 3. Complete details for preparing PCA plots are available.

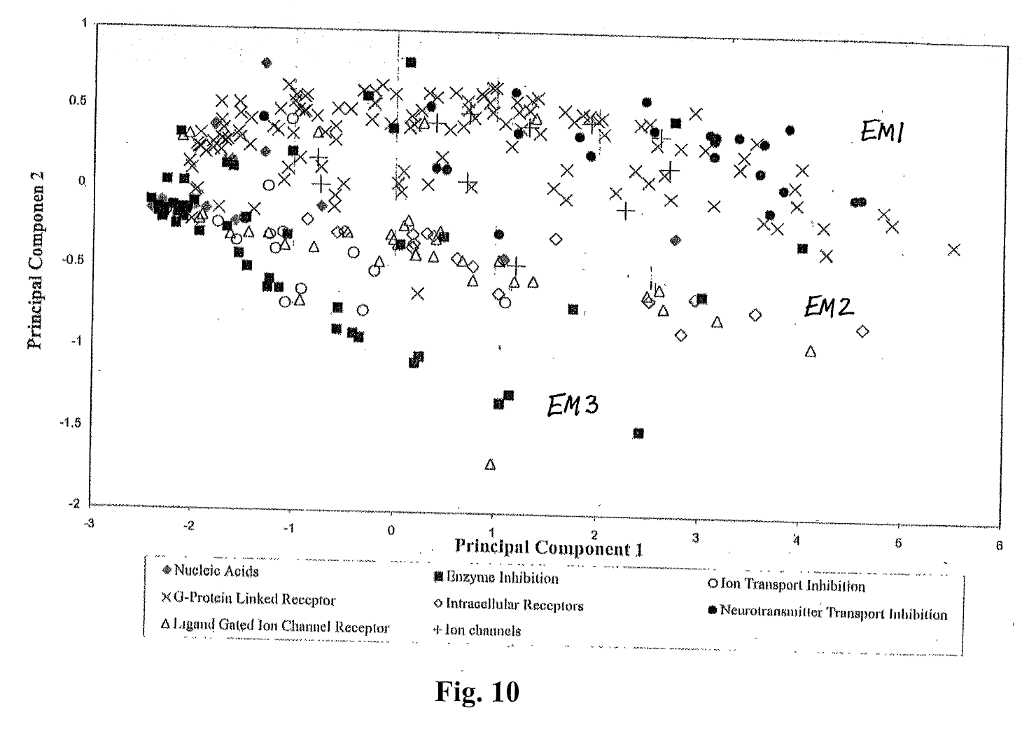

FIG. 10 is a 2-dimensional PCA plot of 400 commercial drugs using 4 membrane binding constants. The graph has three general regions labeled EM-1, EM-2 and EM-3 (EM denotes efficacy mechanisms). Although there are compounds with efficacy mechanisms in a region on the graph that differs from other compounds eliciting the same efficacy mechanisms, this does change the overall trends shown in FIG. 10. As shown in FIG. 10, the EM-1 region has compounds that elicit therapeutic activity using G-coupled receptor proteins, ion channels, and neurotransmitter transport inhibition. The EM-2 region contains compounds that act at intracellular receptors, ligand-gated ion channel receptors, and ion transport inhibitors, and the EM-3 region has compounds that act predominantly as enzyme inhibitors. The three EM regions converge where nucleic acid type compounds reside on the graph. Thus, nucleic acids cannot be assigned to any particular region. In accordance with the present invention, FIG. 10 demonstrates that membrane-binding constants can be used to group compounds according to their efficacy mechanism.

An interesting aspect of FIG. 10 is the overlap among compounds with different efficacy mechanisms. For instance, the EM-1 curve includes both G-protein receptors and neurotransmitter transporters. This means that compounds can elicit different efficacy mechanisms yet have identical membrane binding properties. In other words, compounds with identical membrane binding constants may act at either G-protein receptors or neurotransmitter transporters. Virtually all classification schemes exhibit some level of overlap of the features being characterized and efficacy mechanism is thus no exception. The problem of overlapping features is exemplified in Table 1, which compares different features that have been used to classify drug discovery compounds; these include structure, therapeutic use, receptor, and efficacy mechanisms. With the exception of efficacy mechanisms, statistical methods to classify compounds into each of the other groups have previously been described in the art.

It is useful and routine in drug discovery to group compounds with similar features as described in Table 1. This allows drug discovery compounds to be sorted and pursued as drug leads according to favorable pharmacological features. Historically, there is always overlap among the features, probably because virtually all commercial drugs have side effects, which implies that they act at multiple sites. Consider the following 4 pharmacological features: structure, in vivo activity, receptor, and efficacy mechanism. It may be expected that compounds with similar structures have similar in vivo activity. However, as shown in Table 1, mescaline and amphetamine are both structural analogs of phenylethylamine, but mescaline causes hallucinations where amphetamines are stimulants, which are clearly different in vivo activities. Similarly, grouping compounds by therapeutic use also does not guarantee that the compounds within a group will have the same efficacy mechanism. For instance, chlorpromazine and scopolamine are both antiemetic drugs. Chlorpromazine acts at dopamine receptors whereas scopolamine acts upon muscarine receptors. Both are structurally different yet produce the same therapeutic result. These examples illustrate that there is no single method or process for grouping compounds that shows a unique relationship among structure, receptor, therapeutic use, and efficacy mechanism.

TABLE 1
Representative Methods to Classify Drugs.

| Drug | Receptor | Chemical Class | Therapeutic Class |
|---|---|---|---|
| 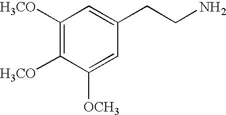 | Serotonin | phenylethylamine | hallucinogen |
| Mescaline | | | |
| 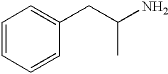 | multiple | phenethylamine | psychostimulant |
| Amphetamine | | | |
| 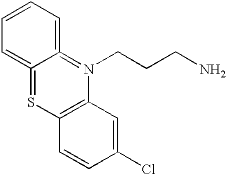 | dopamine | tricyclic | antiemetic |
| Chlorpromazine | | | |
| 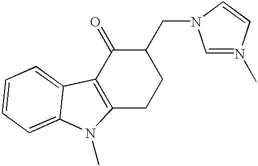 | serotonin | indole | antiemetic |
| Ondansetron | | | |
| 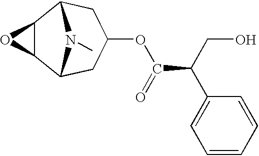 | muscarine | tropine | antiemetic |
| Scopolamine | | | |

The present invention shows that membrane-binding constants can be used to group compounds according to their efficacy mechanism. Equally important is that previous work has demonstrated that membrane-binding constants can group compounds according to their therapeutic use and receptor. Membrane binding is regulated by each compound's structure, as is the intrinsic pharmacological properties of compounds. Thus, the present invention supports the concept that all compound properties are regulated by compound structure, regardless of whether they have been measured or not. The correlation of membrane binding properties with all of the feature groups shown in Table 1 demonstrates the significance of the membrane binding properties of compounds in choosing which compounds to pursue in drug discovery.

The foregoing discussion discloses and describes merely exemplary embodiments of the present invention. One skilled in the art will readily recognize from such discussion, and from the accompanying drawings and claims, that various changes, modifications and variations can be made therein without departing from the spirit and scope of the invention as defined in the following claims.

All references cited herein are incorporated by reference as if fully set forth.

[image]

[image]

[image]

[image]

[image]

[image]

[image]

[image]

[image]

[image]

[image]

[image]

[image]

[image]

What is claimed is:

1. A method for identifying the taxonomic classification of a test drug substance, said method comprising the steps of:
(a) identifying two or more membrane surfaces including a first membrane surface having a negatively charged surface and a second membrane surface that is substantially neutral;
(b) identifying a set of control compounds comprising drug substances having known endogenous macromolecular targets;
(c) defining for each control compound a numeric value related to its affinity for each membrane surface;
(d) defining for the test drug substance a numeric value related to its affinity for each said membrane surface;
(e) identifying a subset of control compounds for each taxonomic target macromolecule wherein the subset has the same or similar target macromolecules and establishing a correlation between said taxonomic target molecule classification and membrane affinity values for te control compounds exhibiting said values; and
(f) comparing the membrane affinity related numeric values for the test drug substance with the taxonomy correlated membrane affinity values for the control compounds; and
(g) selecting the taxonomic classification(s) that best match the membrane affinity values for the test drug substance.

2. The method of claim 1 wherein the membrane surface is selected from Langmuir-Blodgett films, micelles, liposomes and membrane mimetic surfaces.

3. The method of claim 1 wherein the membrane surface is immobilized.

4. The method of claim 1 wherein the negatively charged membrane surface is based on phosphatidylserine and the neutral membrane surface is based on phosphatylethanolamine, phosphatidylcholine or sphingomyelin.

5. The method of claim 1 wherein the negatively charged membrane surface is ^{ester}IAM.PS^{C10/C3} and the neutral membrane mimetic surface is ^{ester}IAM.PE^{C10/C3}, ^{ester}IAM.PC^{C10/C3}, or IAM.SM^{C10/C3}.

6. The method of claim 1 further comprising the steps of:
comparing the structural similarities, based on molecular descriptors, between the test drug substance and the control compounds and identifying those control compounds having a structural similarity to the test drug substance; and
identifying the taxonomic classification for those control compounds having both structural similarity and membrane affinity values similar to those of the test drug substance.

7. A method for identifying the taxonomical classification of a test drug substance, said method comprising the steps of:
(a) identifying two or more membrane surfaces including a first membrane surface having a negatively charged surface and a second membrane surface that is substantially neutral;
(b) identifying a set of control compounds comprising drug substances having known endogenous macromolecular targets;
(c) defining for each control compound a numeric value related to its affinity for each membrane surface;
(d) defining for the test drug substance a numeric value related to its affinity for each said membrane surface; and
(e) comparing the membrane affinity related values for the test drug substance with those for the control compounds and identifying those control compounds having membrane affinity values similar to those of the test compound, and identifying the taxonomical target macromolecule for those control compounds having membrane affinity values similar to those of the test drug substance.

8. The method of claim 7 wherein the membrane surface is selected from Langmuir-Blodgett films, micelles, liposomes and membrane mimetic surfaces.

9. The method of claim 7 wherein the membrane surface is immobilized.

10. The method of claim 7 wherein the negatively charged membrane surface is based on phosphatidylserine and the neutral membrane surface is based on phosphatylethanolamine, phosphatidylcholine or sphingomyelin.

11. The method of claim 7 wherein the negatively charged membrane surface is ^{ester}IAM.PS^{C10/C3} and the neutral membrane mimetic surface is ^{ester}IAM.PE^{C10/C3}, ^{ester}IAM.PC^{C10/C3,}, or IAM.SM^{C10/C3}.

12. The method of claim 7 further comprising the steps of:
comparing the structural similarities, based on molecular descriptors, between the test drug substance and the control compounds and identifying those control compounds having a structural similarity to the test drug substance; and
identifying the taxonomic classification for those control compounds having both structural similarity and membrane affinity values similar to those of the test drug substance.

13. A system for predicting taxonomy of potential endogenous tarted macromolecules of a test drug substance of known chemical structure using a membrane affinity based correlation of the chemical structures of known drug substances, their endogenous target macromolecules, and their empirically defined chemical structure/membrane affinity relationships, said system comprising:
(a) a data storage device having in computer readable format a data set comprising chemical structures of a multiplicity of control compounds comprising drug substances, and for each compound, numeric values relating to the identity or function of the known endogenous macromolecular target, if any, of said compound;
(b) a data entry device for entering the chemical structure of the test drug substance in computer readable form;
(c) a programmable microprocessor in communication with said data entry device and said data storage device, said microprocessor programmed to compare the chemical structure of the test drug substance entered into the data entry device with chemical structures in the data set to identify the chemical structures in the data set having a predetermined degree of similarity with the structure of the test drug substance;
(d) an output device in communication with the microprocessor and capable of reporting, upon user request, the chemical structures or other identification of control compounds having the predetermined degree of similarity with the chemical structure of the test drug substance, and other data stored for said identified control compound(s).

14. The system of claim 13 wherein the chemical structures are stored in two-dimensional format.

15. The system of claim 13 wherein the chemical structures are stored in three-dimensional format.

16. The system of claim 13 wherein the microprocessor is programmed to identify control compounds having membrane binding characteristics similar to the membrane binding characteristics of the control compounds identified to have the predetermined degree of structure similarity to that of the test drug substance.
